(19) United States
(12) Patent Application Publication
(10) Pub. No.: US 2006/0253025 A1
(43) Pub. Date: Nov. 9, 2006

(54) Ultrasonic Bone Assessment Apparatus and Method

(76) Inventor: Jonathan J. Kaufman, Brooklyn, NY (US)

Correspondence Address: DYKEMA GOSSETT PLLC, 39577 WOODWARD AVENUE, SUITE 300, BLOOMFIELD HILLS, MI, 48304-5086, US

(21) Appl. No.: 11/379,454

(22) Filed: Apr. 20, 2006

Related U.S. Application Data
(60) Provisional application No. 60/674,397, filed on Apr. 21, 2005.
(60) Provisional application No. 60/674,167, filed on Apr. 22, 2005.

(51) Int. Cl. A61B 8/00

(52) U.S. Cl. 600/437

(57) ABSTRACT

An invention is disclosed for the assessment of various properties of bone. A method and apparatus are disclosed that use a pair of non-contact (i.e., air-coupled) ultrasound transducers. The invention includes a pair of non-contact ultrasound transducers placed above skin on opposite sides of the bone and generating an ultrasound signal and directing the signal through both the bone to obtain a bone output signal. The method further includes establishing a set of parameters associated with the bone output signal and then further processing the parameters in order to obtain the desired bone property. Apparatuses for the assessment of various properties of bone are also provided. The apparatus includes a pair of ultrasound transducers which may be single-element transducers, focused, or array transducers in any combination. The apparatus further includes various computer hardware components and computer software for generating and directing the ultrasound signal, establishing the parameter set and performing the processing.

### RELATED U.S. APPLICATIONS

This application claims priority to pending U.S. Provisional Patent Application Ser. No. 60/674,397, filed Apr. 21, 2005, the entire disclosure of which is incorporated herein by reference and claims priority to pending U.S. Provisional Patent Application Ser. No. 60/674,167, filed Apr. 22, 2005, the entire disclosure of which is incorporated herein by reference.

### FIELD OF THE INVENTION

The present invention relates generally to apparatus and method for non-invasively and quantitatively evaluating bone tissue in vivo. More specifically, the invention pertains to osteoporosis diagnosis, bone fracture risk assessment, and bone fracture diagnosis using an ultrasound apparatus and method.

### BACKGROUND OF THE INVENTION

In recent years, ultrasound has received a great deal of attention as a new technique for noninvasive assessment of bone, and numerous attempts have been made to use ultrasound energy for evaluating the condition of bone tissue in vivo, and thus for determining a measure of osteoporosis and assessing bone fracture risk.

In particular, Hoop discloses in U.S. Pat. No. 3,847,141 a device to measure bone density as a means for monitoring calcium content of the involved bone. A pair of opposed ultrasonic transducers is applied to opposite sides of a subject's finger, such that recurrent pulses transmitted via one transducer are ‘focused” on the bone, while the receiver response of the other transducer is similarly “focused” to receive pulses that have been transmitted through the bone. The circuitry in Hoop is arranged such that filtered reception of one pulse triggers the next pulse transmission; the filtering is by way of a bandpass filter, passing components of received signals in the 25 kHz to 125 kHz range only; and the observed frequency of retriggering is believed to be proportional to the calcium content of the bone. Thus Hoop is concerned only with what he defines to be transit time for pulses in the indicated band.

Pratt, Jr. deals with establishing, in vivo, the strength of bone in a live being such as a horse. In U.S. Pat. No. 4,361,154, the inventor solves the problem posed by measuring transit time from “launch” to “reception” of pulses of 0.5 MHz and 1.0 MHz through the bone and soft tissue, and from measurement of pulse-echo time, to thereby derive a measurement of transit time through bone alone. A data bank enables the evaluation of the bone condition from the measured transit times. U.S. Pat. No. 4,913,157, also granted to Pratt, Jr., operates on the same general principle of transit time/velocity deduction, using the latter preferred frequency of 2.25 MHz as the base frequency of pulsed “launchings” and a technique of matched filtering/Fourier transform filtering for further analyzing received pulses.

Palmer et al. disclose in U.S. Pat. No. 4,774,959 a bone measurement system deriving the slope of the relation between ultrasonic frequency and attenuation of a sequence of tone signals. Being in the range of 200 kHz to 600 kHz, the signals are applied to one transducer and received by another transducer. The passage of the signals between the two transducers with and without the intervening presence of a heel bone is compared, with the assumption that the frequency/attenuation relation is a straight line, i.e., of constant slope.

U.S. Pat. No. 4,926,870 granted to Brandenburger discloses another in vivo bone analysis system which depends upon measuring transit time for an ultrasonic signal along a desired path through bone. A “canonical” waveform, determined by previous experience to be on the correct path, is used for comparison against received signals for transmission through the patient's bone, while the latter is reoriented until the received signal indicates that the bone is aligned with the desired path. Again, ultrasonic velocity through the patient's bone is assumed to have been determined from measured transit time.

Rossman et al. disclose in U.S. Pat. No. 5,054,490 an ultrasound densitometer for measuring physical properties and integrity of bone, upon determination of a transit time through bone. Alternatively, the Rossman et al. device compares absolute attenuation of specific frequency components of ultrasound signals through the bone with the absolute attenuation of the same frequency components through a medium of known acoustic properties.

Mele et al., disclose in U.S. Pat. No. 5,564,423, and in a subsequent related Patent by Cadossi et al. (U.S. Pat. No. 6,436,042), disclose a device that measures the “amplitude dependent speed of sound” through a bony member in a living body. The method relies on the visual display of the received ultrasound signal, and the selection of a specific portion of the waveform for analysis.

Significant steps in advancing ultrasound bone assessment have been made by Kaufman et al. (in U.S. Pat. Nos. 5,259,384 and 5,651,363) and by Chiabrera et al. (in U.S. Pat. Nos. 5,785,656 and 5,879,301). In these Patents, an estimate of a “bone transfer function” associated with a given bone is obtained in a statistically optimal fashion, and parametric estimates of the phase and attenuation functions associated with it are determined. The disclosed methods also describe the use of 2D array transducers for obtaining more reproducible estimates of the bone density, architecture, and fracture risk.

Notwithstanding the advances made in the past in previous attempts, as exemplified by the above-mentioned apparatuses and methods, there are still additional improvements needed in order to make ultrasound assessment a widely used technique for accurately and precisely assessing the bone density, architecture, quality, fracture diagnosis, and fracture risk of a subject.

### SUMMARY OF THE INVENTION

It is accordingly a primary object of this invention to provide an improved method and apparatus for characterizing and determining non-invasively the properties of bone. A more particular though not limiting object of the invention is to provide a method and apparatus for non-invasive and quantitative evaluation of bone tissue in vivo, to make accurate osteoporosis diagnosis and monitoring possible.

Another object is to meet the above objects in such a way that the bone tissue evaluation and the osteoporosis diagnosis may be performed in a much more convenient manner than those previously used.

A further object is to meet the above object in such a way that the bone tissue evaluation and the osteoporosis diagnosis may be performed with relatively more simple and inexpensive means than those previously used.

A yet further object is to provide an ability to diagnose the presence of a fracture, to monitor the degree of healing, and to assess the osteoporotic fracture risk associated with a given living being.

As compared with the prior art, the invention utilizes air-coupled (i.e., non-contact) ultrasound transducers that is crucial to achieving the indicated objectives. In particular, the present invention uses neither a fluid (e.g., water) bath, or ultrasound coupling gel or gel pad or bag, or any type of “bladder” to acoustically couple the transducer to skin of an interrogated subject. Instead, use is made of presently available air-coupled transducers that makes the entire process much more convenient and attractive for a wide spectrum of users. Moreover, the present invention is based on using a broad set of ultrasound parameters, as well as imaging methods that together is far superior to the approaches disclosed in the prior art.

Accordingly, the present invention utilizes a new configuration to construct an image of an interrogated bony member of a living being, to more conveniently, accurately and precisely determine the characteristics of the interrogated bone—to thereby determine one or more of the bone properties such as fracture risk, strength, density, quality, and/or architecture of the bone. The advantage of such an approach is its inherent simplicity and convenience, as well as its increased sensitivity to the underlying state of the interrogated bone. This is in contrast to the prior art which can not extract as much information on the underlying bone in such a convenient and effective manner.

The invention in its presently preferred form of a method of non-invasive and quantitative assessment of the status of a bone tissue in vivo for one or more of the quantities: bone-mineral density, architecture, strength, quality, and fracture risk, achieves the foregoing objectives by placing a first air-coupled (i.e., non-contact) transducer and a second air-coupled (i.e., non-contact) transducer over nearby skin surfaces (wherein said first and second transducers are not in actual contact with said skin) on opposite sides of a bony member; generating an ultrasound signal and directing this signal from said first transducer to said second transducer through the bone tissue, thereby producing a bone-oriented output signal, the excitation signal being a finite-duration signal repeated substantially in a range from 1 to 5000 Hz and consisting of plural frequencies spaced in an ultrasonic spectral region up to about 5 MHz; and processing the bone-oriented output signal to obtain an estimate of the one or more said quantities.

The step of processing may be performed with the use of one or more of a plurality of associated parameters: age, sex, fracture history, cigarette smoking history, height, and weight that is specific to an individual subject, as well as the thickness of the bony member. In addition, the thickness of the bony member may be determined through use of reflection or pulse-echo measurements. The step of processing may be performed with the use of multivariate linear and nonlinear regressions, a statistical hypothesis testing algorithm, and may include a neural network configured to generate an estimate of the one or more of the quantities from the parameters and from the associated parameters specific for an individual subject.

### BRIEF DESCRIPTION OF THE DRAWINGS

FIG. 1 is a block diagram showing the interconnections of components of an apparatus of the invention.

### DETAILED DESCRIPTION OF THE PREFERRED EMBODIMENT

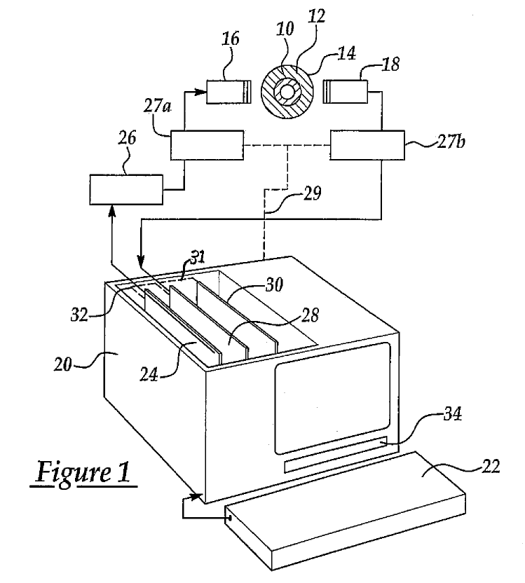

The invention is shown in FIG. 1 as applied to interconnected components for constructing an apparatus for practicing a method of the invention. Specifically, it is intended for non-invasively and quantitatively evaluating the status of bone tissue in vivo, as manifested through one or more of the quantities: bone-mineral density, architecture, quality, strength, and fracture risk at a given time. The components of the apparatus are, in general, commercially available from different sources and will be identified before or in the course of the detailed description of their total operation.

Referring to FIG. 1, a bone 10 to be analyzed in vivo is shown surrounded by a soft tissue 12 having an outer skin surface (skin integument) 14. The bone 10 is to be interposed between two aligned and opposed air-coupled (non-contact) ultrasonic transducers 16 and 18, which may be identically the same, and can be obtained from MicroAcoustic Instruments Inc., located in Ottawa, Ontario, Canada. In the presently preferred embodiment, the air-coupled transducers are focused and are capacitance transducers. As shown, the transducer 16 is used for signal launching and the transducer 18 is the receiver for the launched signals after passing through the bone 10, its surrounding soft tissue 12, and surrounding air between each transducer face and the outer skin surface 14 of the soft tissue 12.

Basic operation is governed by a computer means 20, which may be a personal computer, such as the Dell Precision Workstation 670 available from Dell, Inc., Round Rock, Tex.; this computer contains an Intel Xeon Processor running at 3.60 GHz, with provision for keyboard instruction at 22.

An arbitrary-function generator card 24 is shown installed in the computer 20. This card is relied upon to generate an excitation signal which is periodically supplied to the launch transducer 16, via a power amplifier means 26. The power amplifier 26 is suitably the Model No. 240L, an RF power-amplifier product of EIN, Inc., Rochester, N.Y. This amplifier provides a 50 dB gain, over the range 20 kHz to 10 MHZ. In addition to power amplifier means 26, the excitation signal must pass through a switching network 27a in an alternative embodiment using multi-element, linear- or two-dimensional array transducers, described fully infra.

The excitation signal generated by the card 24 is a finite-duration pulse that is repeated substantially in the range from 1 to 5000 Hz. The card 24 may suitably be a waveform synthesizer, a product of PC Instruments, Inc., Lawrence, Kans., identified by PC Instruments part No. PCI-341. The waveform synthesizer provides generation of analog signals independent of the host computer 20, that allows full processor power to be used for other tasks, including calculation of waveform data.

Another card 28 is shown installed into the computer 20 for converting signals received at the receiving transducer 18 into a digital format for further processing in the computer 20. The card 28 may suitably be a 200 MHZ 14 bit waveform digitizer, a part known as Compuscope 14200, a product available from Gage Applied Technologies, Inc., of Montreal, Quebec, Canada, and which is connected via a cable 32 for electronic timing purposes to arbitrary function card 24. The card 28 includes a number of a levels of amplification; however it should be understood that additional (e.g., external) amplification may be needed and is considered to be within the scope of the present invention.

As with the launch transducer 16, in an alternative embodiment described more fully infra, where receiving transducer 18 is a multi-element, linear or two-dimensional array transducer, a switching network 27b must be placed between the receiving transducer 18 and the card 28 of computer 20. Computer control of the switching networks 27a and 27b is provided via wire connection 29 to a serial (RS 232) port of the computer 20.

Also, general signal-processing/display/storage software, for the signal processing control and operation of the computer 20 is not shown but will be understood to be loaded at CD drive 34 into the computer 20; this software is suitably MATLAB for Windows, available from The Math Works, Inc., Natick, Mass. Further software, also not shown but loaded into the computer 20, are the Neural Network and Optimization Toolboxes, also from The Math Works as well as software (drivers) for interfacing the cards in the computer, available from Gage Applied Technologies and PC Instruments. In addition, a Visual C++ compiler, preferably one available from Microsoft Corporation (Redmond, Wash.) is also understood to be loaded into the computer 20.

In the presently preferred embodiment, involving the described components of FIG. 1, the same components are utilized not only for performing the continuously updated averaging of the latest succession of signals received at the receiving transducer 18 after they have passed through a bone member 10-12-14, but also for establishing and entering into computer storage the data of a reference signal that is obtained by removing the body member 10-12-14 from the space between the transducers 16, 18 and replacing it with a medium with known acoustic properties, such as water, and known path length. This latter signal is useful for calibrating a device so that, e.g., different devices produce the same output parameters (such as broadband ultrasound attenuation (BUA), speed of sound (SOS), net time delay (NTD), mean time duration (MTD) or mean frequency (MF) values when measuring the same object (either a calibration object or a bony member). It may also be used in the computation of a “bone transfer function,” as described fully in U.S. Pat. Nos. 5,259,384, 5,651,363, 5,785,656, and 5,879,301 and which are all incorporated by reference hereinto.

In this embodiment, as may be understood the two transducers consist of a first transducer and a second transducer air-coupled to nearby skin on opposite sides of a bony member (i.e., bone tissue surrounded by overlying soft tissue). The arbitrary-function generator card 24 and power amplifier 26 are used to produce an electrical input signal which is applied to the first transducer. This causes an ultrasound signal to be generated and directed from the first transducer to the second transducer through the bone tissue to obtain a bone-oriented output (received) signal. The bone-oriented output signal is then digitized using the A/D card 28 and processed to obtain an estimate of at least one of parameters NTD, MTD, MF, BUA, and SOS, or the bone transfer function. In alternative embodiments of the invention, a standard pulser may be utilized to generate an ultrasound signal in place of an arbitrary function generator. In this case, a pulser, 30, such as the Matec SR-9000 Pulser/Receiver Plug-In Card may be utilized (Matec Instruments, Northborough, Mass.). In a pulser embodiment, the pulser 30 would be connected directly to either the source transducer 16 or to the switching network 27a, without connection to the amplifier 26. A receiver amplifier may first be used to amplify the signal coming from the switcher 27b or directly from the transducer 18 before digitization by analog to digital (A/D) card 28. In one such preferred embodiment, the pulser-receiver card 30 receives the connection from the switcher 27b or receiver transducer 18, and then sends the amplified signal to the digitizing card 28; these connections are indicated schematically by the dashed line 31. Finally, it should be appreciated that any type of electronic means for generating an excitation signal at the source transducer 16 should be considered to be within the scope of the present invention, including but not limited to arbitrary function generation, tone burst, pulse type and coded excitation signals.

In its presently preferred apparatus form, the invention comprises transducer means including a first air-coupled (i.e., non-contact) transducer and a second air-coupled (i.e., non-contact) transducer placed over nearby skin surfaces (wherein said first and second transducers are not in actual contact with said skin surfaces) on opposite sides of a bony member within a living body; means for generating an ultrasound signal and directing said ultrasound signal from said first non-contact transducer to said second non-contact transducer through said bone tissue to obtain a bone-oriented output signal; and means for processing said bone-oriented output signal, whereby an estimate of said at least one of the quantities, bone-mineral density, bone strength, bone fracture risk, bone architecture and bone quality is obtained.

It should be understood that the ultrasound signal is a finite-duration signal consisting of plural frequencies spaced in the ultrasonic spectral region to approximately 5 MHz and being repeated substantially in the range from 1 Hz to 5000 Hz, and further that the processing means are connected for response to the bone-oriented signal received by the said second transducer (i.e., a receiving transducer) and comprise means to provide analog-to-digital sampling and near real-time processing of the bone-oriented output signals, to thereby produce corresponding parameters and means for performing further analysis of the parameters resulting in estimates of bone properties. It should further be understood that in the presently preferred embodiment of the invention that the corresponding parameters are a net time delay (NTD) parameter and a mean time duration (MTD) parameter; however, it should be appreciated that any set of ultrasound parameters may be utilized in the air-coupled (i.e., non-contact) invention. In particular, the methods and apparatuses as disclosed in U.S. Pat. Nos. 5,259,384, 5,651,363, 5,785,656 and 5,879,301 and which are incorporated by reference hereinto should be understood to be applicable to the present invention.

In the presently preferred embodiment of the invention, the air-coupled transducers are both focused. Any air-coupled focused transducers may be utilized, but in the presently preferred embodiment the transducers are suitably air-coupled capacitance transducers available from MicroAcoustic Instruments Inc. (Ottawa, Ontario, Canada). These transducers are described in U.S. Pat. Nos. 5,287,331 and 5,824,908 and which are included by reference hereinto. These transducers offer sensitivity sufficient for bone assessment as described herein. However, it should be appreciated that other air-coupled transducers can also be used, including capacitive micromachined ultrasound transducers (CMUTS) suitably available from Sensant Corp. (San Leandro, Calif.) and also piezoelectric air-coupled transducers, as available from The Ultran Group (Boalsburg, Pa.). The transducers are held in a standard fixture well known in the art which maintains them a fixed distance apart which is sufficient to ensure no contact with a subjects bony member. The fixture is attached to an x-y mechanical stage, that is used to move the transducers over the bony member in order to create an image of the bone and soft tissue that make up the bony member. The image is composed of values of the set of ultrasound parameters (in the presently preferred embodiment this set is the NTD and MTD) at each point that is scanned by the x-y stage.

It should be appreciated that any air-coupled or non-contact transducer may be utilized in the present invention, and that these transducers may be focused or unfocussed, single or multi-element (array) transducers, and that the array may be either 1D, 2D, or annular. It should also be appreciated that the first and second transducers need not be identical; for example one may be an array, and the other a single element. It should therefore be understood that any combination of transducers should be understood to be within the scope of the present invention.

In the present embodiment, the apparatus consists of a first and second focused non-contact transducers, placed around a bony member of a living person, for example the distal forearm. The two transducers are scanned in non-contact mode over the distal forearm, in a region approximately 4 inches by 8 inches (where the 8 inches is associated with the axis of the arm and the 4 inches is associated with the direction perpendicular. (Note that the size of the region is determined by the size of the bony member and bone tissue within said bony member, and the objectives of the specific diagnosis or assessment being made.) The step size utilized in both directions is 1 mm, but in general both the size and step size in each direction may be different from one another and will depend on the bone being measured and assessment objective. The x-y stage and controller may be any suitable x-y system, but in the presently preferred embodiment the system is available from Techno-Isel (New Hyde Park, N.Y.) and consists of two Narrow Profile Slides together with a stepper motor on each, coupled with a C-10 2-Axis Machine Controller, with associated software and mounting plates, brackets and hardware. An ultrasound signal is generated and sent from the source transducer, through the forearm and to the receiving transducer where it is measured and processed. The processing consists of a specialized moment-based computation, which is based on the first arriving portion of the signal. In particular, two (2) parameters are computed from the received bone-oriented signal, the net time delay (NTD) and the mean time duration (MTD) parameters, respectively. The NTD is the difference between the time delay of an ultrasound signal which was propagated through soft tissue only and the time delay of the actual bone-oriented ultrasound signal, i.e., a signal which propagated through the overlying soft tissues containing the bone tissue. It is represented mathematically as NTD=τₛ−τ_{b}, where τₛ is the time delay associated with a signal which had propagated through soft tissue only (but of identical overall thickness as the bony member) and τ_{b} is the time delay associated with a signal which had propagated through the bony member. The MTD is the time span of a given portion of the received bone-oriented signal, and is generally inversely related to the mean frequency of the signal. In the presently preferred embodiment of the invention, both the NTD and MTD are computed using only the first (half) cycle of the received signal, as it has been found that the later portion of the signal is often corrupted by components having little to do with the condition of the bone tissue per se, for example multiple reflections within the overlying soft tissue. Further NTD and MTD are evaluated using a highly robust (from a statistical perspective) approach, namely computations based on zeroth, first, and second moments of the signal. Let these moments be defined, respectively, as M0, M1, and M2. It should be understood that these moments are computed over the first half cycle of the signal, and further that the moments are defined as the integral of the signal squared with respect to the product of the signal with tⁿ, where n=0, 1, 2, corresponding to M0, M1, and M2, respectively. Note that the integration is from tᵢ to t_{f}, where tᵢ is the start of the first half cycle and t_{f} is the end of the first half cycle of the received bone-oriented signal. The time delay, τ_{b}, of the received bone-oriented signal is evaluated according to τ_{b}=M1/M0, and the time delay of the soft tissue only signal, τₛ, is evaluated according to τₛ=d/Vs, where d is assumed equal to the thickness of the bony member, and Vs is the velocity of ultrasound within the soft tissue. Therefore, the NTD=d/Vs−M1/M0. The MTD is evaluated according to MTD=M2/M0−{M1/M0}².

In the presently preferred embodiment of the invention, the bone mineral density (BMD) of the bone in the bony member is evaluated according to a linear regression between NTD and BMD, i.e., BMD=a·NTD+b. The fracture risk associated with the living person is provided by a feedforward neural network whose inputs are the ultrasound parameters NTD and MTD, and the associated parameters age, sex, weight, height and history of fracture. The output of the neural network is the probability of fracture, a number between 0 and 1.

An apparatus for the preceding embodiments is shown in FIG. 1. It should be understood that alternative functions may be utilized for estimating the BMD and fracture risk, not just a linear univariate one (for BMD) and not just a neural network (for fracture risk). For example, BMD can be evaluated using multivariate non-linear regressions, and fracture risk can be evaluated with an analytic model and statistical pattern recognition approaches which can prove useful in certain cases. It should also be appreciated that the invention as herein disclosed may be embodied using at least one of said parameter NTD and MTD.

It should be pointed out that the computation of the net time delay often relies on knowledge of the bony member thickness. In an alternative embodiment of the invention, this can be assessed using pulse echo measurements. In this approach, the distance between the skin surface and transducer are estimated using (½) the time delay of a reflection of the ultrasound wave to return and be detected by the transducer. This time delay, together with knowledge of the speed of ultrasound in air (which in general would be determined in a through-transmission measurement made before or after the measurement of the reflected signal) would allow computation of the distance of the skin from the transducer. Doing this on both sides of the bony member (i.e., operating both transducers in reflection mode) would provide an accurate estimate of bony member thickness at any point. Of course, this presupposes that the distance separating the two transducers is known, which is easily done. An alternative to using pulse-echo measurements, and one in which the bony member thickness is not needed at all is as follows. In this additional preferred embodiment, a soft tissue-oriented signal is used. The soft tissue-oriented signal is obtained by propagating an ultrasound signal through the soft tissue portion only of the bony member. This signal then has associated with it a delay composed of the two delays associated with propagation through air only (that associated with both sides of the bony member) as well as the delay associated with propagation through the soft tissue only portion of the bony member. This can then be used for computation of the NTD; in this case there is no need for using d/Vs, as this is already included in the time delay of the reference signal, and further there is no need for explicit evaluation of bony member thickness, d. However, it should be understood that this approach assumes that the bony member thickness is the same at the places where the soft tissue only and bone tissue measurements are made. The present inventors have found that this is a reasonable approximation in a number of sites, such as the distal forearm.

It should be further understood that this approach can also be utilized in a contact transducer or immersion transducer system, as well. In this case, a presently preferred embodiment utilizes a large single element source transducer and a linear array receiver transducer. A signal which has propagated through only a soft tissue portion is used to compute the soft tissue delay, and is used together with a signal which has propagated through a portion of the bony member containing both bone and soft tissue. In a presently preferred embodiment of the invention, this is located at the “soft-tissue only” space in the forearm between the ulna and radius. This can be described by:

A method of quantitative assessment of the status of bone tissue in a bony member in vivo for at least one of the quantities, bone-mineral density, bone strength, bone fracture risk, bone architecture and bone quality comprising the steps of:

placing a first transducer and a second array transducer on opposite sides of said bony member, wherein said first transducer and said second array transducer are in contact with skin of said bony member;

generating an ultrasound signal and directing said ultrasound signal from said first transducer to said second array transducer through said bone tissue to obtain a set of bone-oriented output signals, wherein said set includes signals propagating through soft tissue only; and

processing said set of bone-oriented output signals whereby an estimate of said at least one of the quantities, bone-mineral density, bone strength, bone fracture risk, bone architecture and bone quality is obtained.

It should therefore be understood that the use of the soft-tissue only portion may be used in both a contact or a non-contact mode, in either an array embodiment or a non-array (scanning) embodiment of the invention. It should also be appreciated that the said second array transducer can be in other embodiments of the invention either a transmitter or a receiver, and that it may be a linear array or a 1.5D or 2D array. In the presently preferred embodiment of the invention, the said second array transducer is a linear array receiver. In particular, the invention comprises a method of quantitative assessment of the status of bone tissue in a bony member in vivo for at least one of the quantities, bone-mineral density, bone strength, bone fracture risk, bone architecture and bone quality comprising the steps of:

placing a first linear array transducer and a second transducer on opposite sides of said bony member, wherein said first transducer and said second array transducer are in contact with skin of said bony member;

generating a set of ultrasound signals and directing said set ultrasound signals from said first array transducer to said second transducer through said bone tissue to obtain a set of bone-oriented output signals wherein said set includes signals propagating through soft tissue only; and

processing said set of bone-oriented output signals whereby an estimate of said at least one of the quantities, bone-mineral density, bone strength, bone fracture risk, bone architecture and bone quality is obtained. In the above embodiment in which an estimate of soft tissue delay time is obtained from processing an ultrasound signal which propagated through soft tissue only, it should be understood that the bone member thickness must be either known or maintained to be a constant. In a presently preferred embodiment, the thickness is maintained as constant by the source and receiver transducers compressing the bony member in a parallel fashion.

It should be further appreciated that the time delay of the soft tissue-oriented signal can be computed using any approach that is suitable, for example the method of moments as disclosed hereinabove for the evaluation of the time delay associated with the bone-oriented reference signal. It should also be understood that while in the presently preferred embodiments of the invention the time delays of the bone-oriented signal and soft tissue-oriented signal are computed with the method of moments, other approaches could be used. These methods could include, but not be limited to selection of the time delay based on when the signal first rises above a noise level, or the time when the signal first reaches 5 percent of its maximum value. Thus the present invention should be understood to include any methods which can estimate a time delay of a signal. It should be further understood that the invention disclosed herein may incorporate other ultrasound parameters, such as broadband ultrasound attenuation, mean frequency, velocity, or any of a variety of measured parameters that are useful for estimating the indicated bone variables.

As yet a further embodiment of the invention it should be appreciated that the use of coded waveforms is useful for improving the signal-to-noise ratio (SNR), which because of the air coupled nature of the invention can often be extremely low. In this regard, the use of coding—and decoding—will improve the SNR so that more reliable estimates of the bone properties can be achieved. Thus, coding as part of the generation of the ultrasound waveforms, and decoding as part of the processing of the received waveforms, can find great utility in the various embodiments of the present invention. A good review of this can be found in the articles included Special Issue on Coded Waveforms in Ultrasonic Imaging [IEEE Transactions on Ultrasonics, Ferroelectrics, and Frequency Control, February 2005, Vol. 52, No. 2], and in the references cited therein, all of which are included by reference hereinto.

It should be also understood that the methods and apparatuses disclosed herein can find application at anatomical sites which are largely trabecular (such as at the heel) or largely cortical (such as at the midshaft of the finger bone or ⅓ of the length up from the distal end of the radius or ulna) or both (e.g., the ultra-distal radius or the proximal or distal phalanx). In addition, the imaging methods are useful for reproducibly locating a region of interest (ROI), and various template matching and edge detection processing, well known in the art, should be understood to be included in the various embodiments of the invention. It should also be pointed out that the transducers may or may not be mechanically scanned; in an alternative to scanning, a phased or unphased, linear, 1.5D or 2D array can be utilized, or a single element transducer pair can be used when placement of the transducers can be reproducibly achieved, as for example at the mid-shaft of a phalanx. In addition, in yet another embodiment, the distance separating the transducers can be changed in order to maintain proper focusing which may be necessary depending upon the properties of the materials through which the ultrasound wave is propagating. This type of adjustment is described in the excellent reference attached hereto (and denoted as “Technical Notes” on pre-numbered pages shown as pp. 32-40 and obtained from the Web link http://wwww.panametrics-ndt.com/ndt/downloads/transducer_technotes.pdf from the web site of Panametrics-NDT, a business of R/D Tech Instruments Inc., located in Waltham, Mass.). Note particularly Eq. 13 in these Technical Notes. It should also be understood that Eq. 13 must be modified in the case of the present invention, namely the incorporation of non-contact or air-coupled transducers. This is done by taking into account the speed of ultrasound in air and the frequency of ultrasound. Additional information in this regard may be found in two excellent references: (1) Acoustic Waves: Devices, Inaging, & Analog Signal Processing, published by Prentice-Hall, Inc., Englewood Cliffs, N.J. in 1987 and written by Gordon S. Kino; and (2)Ultrasonic Bioinstrumentation, published by John Wiley and Sons, Inc., New York, N.Y. in 1988 and written by Douglas A. Christensen, and which are both included by reference hereinto. It should also be appreciated that changing the distance separating the transducers is also useful in a contact mode as well, including but limited to immersion (i.e., water or some other fluid), or other type of contact method (e.g., gel pads). In a presently preferred embodiment of the invention, the distance separating the transducers is controlled by a stepper motor and slide, together with a digital controller, similar to the kind disclosed hereinabove for the x-y slide. However, it should be understood that any method for changing the effective distance separating the transducers can be considered to be within the scope of the present invention, for example, using an annular array capable of having its focus modified thorough appropriate phasing. In this regard, the information contained in Chapters 9 and 10 in the excellent reference Medical Imaging Systems, by Albert Macovski, published by Prentice-Hall, Inc., Englewood Cliffs, N.J. in 1983 is useful and is incorporated by reference hereinto. It should be appreciated that the variations in acoustic properties of the bony member at various locations, or the variations in bony members between subjects, makes the change in focus or focal length necessary in certain instances. This can also be a result of changes in frequency in the received signal because of frequency-dependent attenuation; thus the use of ultrasound transducer pairs at distinct frequencies and focal lengths should be considered to be within the scope of the present inventions, either the non-contact (air-coupled) one, or the contact one.

In a yet further embodiment of the invention and with additional reference to FIG. 2, two pairs of transducers are utilized. The first set is used to identify as accurately as possible the edge (outline) of the bone within the bony member. Following this, another transducer pair (whose location with respect to the first pair is known) is used to measure a specific region of interest of the bone tissue, said region of interest based on the outline of the bone determined using the first transducer pair. Note that this embodiment may be useful when both trabecular and cortical bone want to be examined in the same bony member, for example in the forearm. This could also be achieved through annular arrays which are capable of multiple focusing depths and also the potential for multi-frequency operation. One such embodiment utilizes annular arrays operated at two distinct frequencies, which can be achieved both with a single crystal thickness (and operated at the fundamental and harmonics of the transducer) or by distinct thicknesses in the rings of the annular array, to obtain the desired multi-frequency operation. It should be further appreciated that the various embodiments described herein can be used in the non-contact (air-coupled) transducer bone assessment invention, and in the contact transducer bone assessment invention.

The techniques disclosed herein can also find application in fracture detection and in fracture healing assessment, by providing an image and an assessment of the degree of healing at the fracture site. It should also be understood that the methods and apparatuses disclosed herein can be used not only in humans but in any animal as well, including but not limited to a horse.

It should also be understood that the methods and apparatus disclosed herein can be used to estimate one or more of the said bone quantities, namely, bone density, bone quality, bone architecture, fracture risk, and bone strength not only of the bony member being ultrasonically interrogated, but also at a site anatomically remote to that site, for example, the hip and spine. Further, the methods and apparatuses disclosed herein can be used at any anatomical site, including but not limited to the hips, spine, forearms, fingers, thighs, heels, ribs, and tibia. Moreover, the methods and apparatuses disclosed herein can be used in pulse-echo mode, and the bone quantities can be estimated using backscattered signals, alone or in conjunction with the through transmitted waveforms. In the pulse echo mode, reception of the backscattered signal can be done with the same transducer that transmitted the wave, or with a distinct transducer, or alternatively with distinct elements or the same elements, in the context of a multi-element transducer.

While several embodiments of the present invention have been disclosed hereinabove, it is to be understood that these embodiments are given by example only and not in a limiting sense. Those skilled in the art may make various modifications and additions to the preferred embodiments chosen to illustrate the invention without departing from the spirit and scope of the present contribution to the art. Accordingly, it is to be realized that the patent protection sought and to be afforded hereby shall be deemed to extend to the subject matter claimed and all equivalence thereof fairly within the scope of the invention.

I claim:

1. A method of non-contact and quantitative assessment of the status of bone tissue in a bony member in vivo for at least one of the quantities, bone-mineral density, bone strength, bone fracture risk, bone architecture and bone quality comprising the steps of:
placing a first air-coupled transducer and a second air-coupled transducer on opposite sides of said bony member, wherein said first and second transducers are not in contact with skin of said bony member;
generating an ultrasound signal and directing said ultrasound signal from said first transducer to said second transducer through said bone tissue to obtain a bone-oriented output signal;
processing said bone-oriented output signal whereby an estimate of said at least one of the quantities, bone-mineral density, bone strength, bone fracture risk, bone architecture and bone quality is obtained.

2. The method of claim 1 wherein said bony member is a heel.

3. The method of claim 1 wherein said bony member is a forearm.

4. The method of claim 1 wherein said bony member is a finger.

5. The method of claim 1 wherein said bony member is a thigh.

6. The method of claim 1 wherein said bony member is a calf.

7. The method of claim 1 wherein at least one of said first air-coupled transducer and said second air-coupled transducer is an air-coupled array transducer.

8. The method of claim 1 wherein at least one of said first air-coupled transducer and said second air-coupled transducer is a focused air-coupled transducer.

9. The method of claim 1 wherein at least one of said first air-coupled transducer and said second air-coupled transducer is mechanically translated over said bony member.

10. The method of claim 1 wherein at least one of said first air-coupled transducer and said second air-coupled transducer is operated in pulse-echo mode.

11. The method of claim 1 wherein said generating an ultrasound signal includes ultrasound coding, and wherein said processing includes ultrasound decoding, whereby a signal-to-noise ratio is improved.

12. A method of non-invasive and quantitative assessment of the status of bone tissue in a bony member in vivo for at least one of the quantities, bone-mineral density, bone strength, bone fracture risk, bone architecture and bone quality comprising the steps of:
placing a first air-coupled transducer and a second air-coupled transducer on opposite sides of said bony member, wherein said first and second transducers are not in contact with skin of said bony member;
generating a ultrasound signal and directing said ultrasound signal from said first transducer to said second transducer through said bone tissue to obtain a bone-oriented output signal;
generating another ultrasound signal and directing said another ultrasound signal from said first transducer to said second transducer through a soft tissue only portion of said bony member to obtain a soft tissue-oriented output signal;
processing said bone-oriented output signal and said soft tissue-oriented output signal whereby an estimate of said at least one of the quantities, bone-mineral density, bone strength, bone fracture risk, bone architecture and bone quality is obtained.

13. The method of claim 12 wherein said bony member is a heel.

14. The method of claim 12 wherein said bony member is a forearm.

15. The method of claim 12 wherein said bony member is a finger.

16. The method of claim 12 wherein said bony member is a thigh.

17. The method of claim 12 wherein said bony member is a calf.

18. The method of claim 12 wherein at least one of said first air-coupled transducer and said second air-coupled transducer is an air-coupled array transducer.

19. The method of claim 12 wherein at least one of said first air-coupled transducer and said second air-coupled transducer is a focused air-coupled transducer.

20. The method of claim 12 wherein at least one of said first air-coupled transducer and said second air-coupled transducer is mechanically translated over said bony member.

21. The method of claim 12 wherein at least one of said first air-coupled transducer and said second air-coupled transducer is operated in pulse-echo mode.

22. The method of claim 12 wherein said generating an ultrasound signal includes ultrasound coding, and wherein said processing includes ultrasound decoding, whereby a signal-to-noise ratio is improved.

23. An apparatus for non-contact and quantitative assessment of the status of bone tissue in a bony member in vivo for at least one of the quantities, bone-mineral density, bone strength, bone fracture risk, bone architecture and bone quality comprising:
first and second non-contact transducers including means for placing said first and second non-contact transducers nearby skin on opposite sides of said bony member;
means for generating an ultrasound signal and directing said ultrasound signal from said first non-contact transducer to said second non-contact transducer through said bone tissue to obtain a bone-oriented output signal; and
means for processing said bone-oriented output signal, whereby an estimate of said at least one of the quantities, bone-mineral density, bone strength, bone fracture risk, bone architecture and bone quality is obtained.

24. An apparatus for non-contact and quantitative assessment of the status of bone tissue in a bony member in vivo for at least one of the quantities, bone-mineral density, bone strength, bone fracture risk, bone architecture and bone quality comprising:
first and second non-contact transducers including means for placing said first and second non-contact transducers nearby skin on opposite sides of said bony member;
means for generating an ultrasound signal and directing said ultrasound signal from said first non-contact transducer to said second non-contact transducer through said bone tissue to obtain a bone-oriented output signal;
means for generating another ultrasound signal and directing said ultrasound signal from said first non-contact transducer to said second non-contact transducer through a soft tissue only portion of said bony member to obtain a soft tissue-oriented output signal; and
means for processing said bone-oriented output signal and said reference output signal, whereby an estimate of said at least one of the quantities, bone-mineral density, bone strength, bone fracture risk, bone architecture and bone quality is obtained.

25. A method of quantitative assessment of the status of bone tissue in a bony member in vivo for at least one of the quantities, bone-mineral density, bone strength, bone fracture risk, bone architecture and bone quality comprising the steps of:
placing a first transducer and a second linear array transducer on opposite sides of said bony member, wherein said first transducer and said second array transducer are in contact with skin of said bony member;
generating an ultrasound signal and directing said ultrasound signal from said first transducer to said second array transducer through said bone tissue to obtain a set of bone-oriented output signals, wherein said set includes signals propagating through soft tissue only; and
processing said set of bone-oriented output signals whereby an estimate of said at least one of the quantities, bone-mineral density, bone strength, bone fracture risk, bone architecture and bone quality is obtained.

26. A method of quantitative assessment of the status of bone tissue in a bony member in vivo for at least one of the quantities, bone-mineral density, bone strength, bone fracture risk, bone architecture and bone quality comprising the steps of:
placing a first linear array transducer and a second transducer on opposite sides of said bony member, wherein said first transducer and said second array transducer are in contact with skin of said bony member;
generating a set of ultrasound signals and directing said set ultrasound signals from said first array transducer to said second transducer through said bone tissue to obtain a set of bone-oriented output signals, wherein said set includes signals propagating through soft tissue only; and
processing said set of bone-oriented output signals whereby an estimate of said at least one of the quantities, bone-mineral density, bone strength, bone fracture risk, bone architecture and bone quality is obtained.
